(19) United States
(12) Patent Application Publication
(10) Pub. No.: US 2023/0405114 A1
(43) Pub. Date: Dec. 21, 2023

(54) MYCOBACTERIAL IMMUNOTHERAPY FOR TREATING CANCER

(71) Applicant: IMMODULON THERAPEUTICS LIMITED, LONDON (GB)

(72) Inventors: GLEN MARTYN, UXBRIDGE (GB); JAKOB KAMPINGA, UXBRIDGE (GB); THOMAS KLEEN, UXBRIDGE (GB)

(21) Appl. No.: 18/252,048

(22) PCT Filed: Nov. 5, 2021
(86) PCT No.: PCT/GB2021/052881

§ 371 Date: May 8, 2023

(30) Foreign Application Priority Data
Nov. 6, 2020 (GB) 2017554.3
Mar. 29, 2021 (GB) 2104427.6

(51) Int. Cl. A61K 39/39; A61K 39/395; A61P 35/00

(52) U.S. Cl. CPC A61K 39/39; A61K 39/3955; A61P 35/00; A61K 2039/521

(57) ABSTRACT

This invention provides an immunomodulator for use in the adjuvant, neoadjuvant or peri-adjuvant treatment, reduction, inhibition or control of cancer in patients that have undergone, or are intended to undergo, tumour resection surgery and/or one or more additional anticancer treatments or agents, preferably checkpoint inhibitor therapy. The invention also describes the use of one or more additional anticancer treatments or agents in the adjuvant, neoadjuvant or peri-adjuvant setting, as well as protocols and dosage regimens for use.

### FIELD OF INVENTION

The present invention relates to the field of cancer therapy. In particular, the present invention relates to a method of preventing, treating or inhibiting the development of tumours and/or metastases in a subject through adjuvant therapies, including neo-adjuvant, adjuvant and peri-adjuvant regimens and therapies.

### BACKGROUND

In humans with cancer, treatment regimens often include forms of oncological surgical intervention, chemo-, radio- or immuno-therapies. In advanced presentations cancer, anti-tumour immunity is often ineffective due to the tightly regulated interplay of pro- and anti-inflammatory, immune-stimulatory and immunosuppressive signals. It is now believed that the immune system constantly monitors and eliminates newly transformed cells. Accordingly, cancer cells may alter their phenotype in response to immune pressure in order to escape attack (immunoediting) and upregulate expression of inhibitory signals. Through immunoediting and other subversive processes, primary tumour and metastasis maintain their own survival.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (TIM-3), Lymphocyte Activation Gene-3 (LAG-3; CD223), and others are often referred to as a checkpoint regulators. They act as molecular “tollbooths,” which allow extracellular information to dictate whether cell cycle progression and other intracellular signalling processes should proceed.

In addition to specific antigen recognition through the T-cell receptor (TCR), T-cell activation is regulated through a balance of positive and negative signals provided by co-stimulatory receptors. These surface proteins are typically members of either the TNF receptor or B7 superfamilies. Agonistic antibodies directed against activating co-stimulatory molecules and blocking antibodies against negative co-stimulatory molecules may enhance T-cell stimulation to promote tumour destruction.

Two ligands specific for PD-1 have been identified: programmed death-ligand 1 (PD-L1, also known as B7-H1 or CD274) and PD-L2 (also known as B7-DC or CD273). PD-L1 and PD-L2 have been shown to down-regulate T cell activation upon binding to PD-1 in both mouse and human systems (Okazaki et al., Int Immunol., 2007; 19: 813-824). The interaction of PD-1 with its ligands, PD-L1 and PD-L2, which are expressed on antigen-presenting cells (APCs) and dendritic cells (DCs), transmits negative regulatory stimuli to down-modulate the activated T cell immune response. Blockade of PD-1 suppresses this negative signal and amplifies T cell responses.

The PD-L1/PD-1 signalling pathway is a primary mechanism of cancer immune evasion for several reasons. First, and most importantly, this pathway is involved in negative regulation of immune responses of activated T effector cells, found in the periphery. Second, PD-L1 is up-regulated in cancer microenvironments, while PD-1 is also up-regulated on activated tumour infiltrating T cells, thus possibly potentiating a vicious cycle of inhibition. Third, this pathway is intricately involved in both innate and adaptive immune regulation through bi-directional signalling. These factors make the PD-1/PD-L1 complex a central point through which cancer can manipulate immune responses and promote its own progression.

The first immune-checkpoint inhibitor to be tested in a clinical trial was ipilimumab (Yervoy, Bristol-Myers Squibb), a CTLA-4 mAb. Anti-CTLA-4 mAb is a powerful checkpoint inhibitor which removes “the break” from both naïve and antigen-experienced cells. Therapy enhances the antitumor function of CD8+ T cells, increases the ratio of CD8+ T cells to Foxp3+ T regulatory cells, and inhibits the suppressive function of T regulatory cells. The major drawback to anti-CTLA-4 mAb therapy is the generation of autoimmune toxicities.

TIM-3 has been identified as another important inhibitory receptor expressed by exhausted CD8+ T cells. In mouse models of cancer, it has been shown that the most dysfunctional tumour-infiltrating CD8+ T cells actually co-express PD-1 and TIM-3.

LAG-3 is another recently identified inhibitory receptor that acts to limit effector T-cell function and augment the suppressive activity of T regulatory cells. It has recently been revealed that PD-1 and LAG-3 are extensively co-expressed by tumour-infiltrating T cells in mice, and that combined blockade of PD-1 and LAG-3 provokes potent synergistic antitumor immune responses in mouse models of cancer.

Immune checkpoint inhibitor therapy has been particularly successful in melanoma, for which approved treatments now include anti-PD-1 (nivolumab and pembrolizumab), anti-CTLA-4 (ipilimumab), and combination anti-PD-1/CTLA-4 regimens (nivolumab-ipilimumab). Long-term survival data for patients with melanoma treated with ipilimumab (antiCTLA-4) indicates 20% of patients show evidence of continued durable disease control or response 5-10 years after starting therapy. The response rate for melanoma patients treated with pembrolizumab (anti-PD-1) was 33% at 3 years with 70-80% of patients initially responding maintaining clinical response.

Yet whilst immunotherapy continues to advance, as well as the management of cancer through surgical interventions, many subjects still face an extremely poor prognosis. Mismatch repair-deficient (dMMR) cancer with microsatellite instability (MSI), for example, remains problematic, and is particularly prevalent in elderly patients due to a higher rate of methylated hMLH1 gene promoters. Such dMMR cancers are also typical of colorectal cancers, given that the median age of patients with newly diagnosed colorectal cancer is greater than 70 years. In the German COLOPREDICT registry the rate of dMMR tumours is up to 25% in stage II and 20% in stage Ill colorectal cancer. The standard of care in stage III colorectal cancer is currently oxaliplatin and fluoropyrimidine-based adjuvant chemotherapy. However, for many patients, including those over the age of 75, oxaliplatin is not recommended by several guidelines and is often not feasible due to underlining comorbidities in elderly patients (Nitsche et al. 2017, Gastrointest Tumours 4(1-2): 11-19). The relapse rate in patients with stage III colorectal cancer not treated with oxaliplatin-based adjuvant chemotherapy is relatively high, with a 3-year disease-free survival-rate and a 5-year overall survival rate of only 60% and 67%, respectively (Gill et al. 2014, Indian J Med Paediatr Oncol. 35(3): 197-202).

As such, there exists a need for an alternative therapeutic option to improve the unfavourable prognosis of patients with stage III dMMR colorectal cancer ineligible for oxaliplatin-based adjuvant chemotherapy, and indeed the prognoses of patients with other difficult to treat and/or high relapse rate cancers.

Accordingly, an aim of the present invention is to provide a therapy for treating cancer in patients undertaking tumour resection surgery or checkpoint inhibitor therapy. This therapy comprises a non-viable Mycobacterium, used as a neo-, peri- or adjuvant agent or therapy, with the aim of maximising the effectiveness of surgery or checkpoint inhibitor therapy.

### SUMMARY OF INVENTION

The present invention provides an effective method for treating, reducing, inhibiting, controlling and/or preventing cancer and/or the establishment of metastases, in a patient that has undergone, or is intended to undergo, tumour resection surgery administration of one or more additional anticancer treatments or agents, preferably checkpoint inhibitor therapy, by administering an immunomodulator following and/or prior to said surgery or checkpoint immunotherapy, comprising a non-pathogenic, non-viable, Mycobacterium.

Thus, in a first aspect of the invention, there is provided an immunomodulator for use in the adjuvant, neoadjuvant or peri-adjuvant treatment, reduction, inhibition or control of cancer comprising a primary tumour in a patient that has undergone, or is intended to undergo, tumour resection surgery and administration of one or more additional anticancer treatments or agents, preferably checkpoint inhibitor therapy, wherein said immunomodulator comprises non-pathogenic non-viable Mycobacterium, optionally wherein said patient does not present with metastases in any organs distant to the primary tumour.

In a second aspect of the invention, there is provided a method of treating or controlling cancer comprising a primary tumour in a patient that has undergone, or is intended to undergo, tumour resection surgery, wherein said method comprises simultaneously, separately or sequentially administering to the subject, (i) a non-pathogenic non-viable Mycobacterium, (ii) tumour resection surgery, and (iii) one or more additional anticancer treatments or agents, wherein said method results in enhanced therapeutic efficacy relative to administration of non-pathogenic non-viable Mycobacterium, administration of one or more additional anticancer treatments or agents, or tumour resection surgery alone.

### DESCRIPTION OF THE DRAWINGS

The invention is described with reference to the following drawings, in which:

FIG. 1 shows a schematic of a clinical trial investigating the adjuvant (post-surgical) use of a combination according to the invention, specifically M. obuense with the checkpoint inhibitor (anti-PD-L1) atezolizumab, in the treatment of Stage III, R0 resected, MSI-high/dMMR patients, who are ineligible for oxaliplatin or refused said agent, and who present with an ECOG Performance Status of 0, 1 or 2 (see Example 3).

FIG. 2 shows a schematic/flow chart of a clinical trial investigating the neo-adjuvant (pre-surgical) use and adjuvant (post-surgical) use, together a peri-adjuvant regimen, of a combination according to the invention, specifically M. obuense with the checkpoint inhibition (anti-PD-L1) atezolizumab, in the treatment of Stage III, resectable MSI-high/dMMR patients, who are ineligible for oxaliplatin or refused said agent, and who present with an ECOG Performance Status of 0, 1 or 2 (see Example 4).

### DETAILED DESCRIPTION OF THE INVENTION

The invention provides a method for treating, reducing, inhibiting or controlling a neoplasia, tumour or cancer in a subject undertaking tumour resection surgery or immunotherapy involving administering a non-pathogenic, non-viable, Mycobacterium and optionally one or more chemotherapy agents. It is based upon the surprising discovery that administration of a non-pathogenic heat-killed Mycobacterium as a neoadjuvant treatment prior to surgery or checkpoint inhibitor therapy (CPI), optionally in combination with further chemotherapy agents, results in enhanced anti-tumour activity and/or antitumor activity that is more potent than undertaking the surgery or CPI alone. Further, treatment with the Mycobacterium was shown to result in an effective and sustained pathological response and survival of treated patients.

In a phase II clinical trial involving patients with pancreatic ductal adenocarcinoma (PDAC), it was found that administering non-pathogenic heat-killed Mycobacterium in a multimodality neoadjuvant treatment regimen combined with the chemotherapy agent gemcitabine, resulted in a surprisingly effective and sustained pathological response and survival relative to gemcitabine alone, particularly in metastatic patients.

Accordingly, whilst some neoadjuvant therapies are known in the art to aid resection surgeries or other cancer treatments, the invention disclosed herein provides specific immunomodulator-based neo, peri- and adjuvant protocols which are optimised to improve therapeutic efficacy and thus responses in a greater proportion of subjects that undergo surgery and/or checkpoint immunotherapy.

In order that the present invention may be more readily understood, certain terms are first defined. Additional definitions are set forth throughout the detailed description.

The term “neoadjuvant” or “neoadjuvant therapy”, which may be used interchangeably with “preoperative therapy”, refers to the administration of one or more therapeutic agents or modalities prior to a main treatment or surgery. It is the aim of such a therapy to effectively reduce the difficulty and morbidity of more extensive surgical procedures, as well as enhance the overall efficacy of said surgical procedures.

The terms “adjuvant therapy” and “peri-adjuvant therapy” are also used herein, which refer to therapies administered during or preferably after surgery or treatment, or, both prior to and after/following surgery or treatment, respectively.

A “tumour resection surgery”, which may be referred to herein simply as “surgery” or “surgical procedure”, refers to procedures that aim to physically remove all or part of a tumour, preferably a primary tumour or node, or isolated, single tumour or node. Tumour resection surgery is often used before chemotherapy or radiation, and often in a regimen involving adjuvant therapy. The term tumour resection surgery is used to define a variety of forms of tumour resection surgeries, and may further include ancillary surgeries, including those of lymph node resection nature, such as lymphadenectomy. It may also encompass surgery on a localised metastatic tumour.

A “checkpoint inhibitor” is an agent which acts on surface proteins which are members of either the TNF receptor or B7 superfamilies, including agents which bind to negative co-stimulatory (co-inhibitory) molecules selected from CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3, and/or their respective ligands, including PD-L1. A “blocking agent” is an agent which either binds to the above negative co-stimulatory molecules and/or their respective ligands. “Checkpoint inhibitor” and “blocking agent” are used interchangeably throughout.

Response evaluation criteria in solid tumours (under RECIST) forms the basis for progression free survival (PFS) determination and defines progressive disease as at least a 20% increase in the sum of diameters of up to 5 target lesions (2 lesions/organ), taking as reference the smallest sum on study and an absolute lesion increase of at least 5 mm or the appearance of new lesions. A complete response is the disappearance of all target lesions, and a partial response (PR) is defined as at least a 30% decrease in the sum of the target lesions. Stable disease is defined as fitting the criteria neither for progressive disease nor a PR. PFS is defined as the time from randomization to progression or death. Time to progression is a related, less-preferred end point wherein deaths without progression are censored observations rather than counted as events.

A non-viable, non-pathogenic Mycobacterium as defined according to the present invention, is a component which stimulates innate and type-1 immunity, including Th1 and macrophage activation/polarization and cytotoxic cell activity, as well as independently down-regulating inappropriate anti-Th2 responses via immunoregulatory mechanisms. IMM-101 is an investigational systemic immunomodulator comprising heat-killed Mycobacterium obuense. IMM-101 contains microbial-associated molecular patterns (MAMPs) that activate a defined selection of pathogen recognition receptors (PRRs) including toll like receptor (TLR) ½ on innate immune cells like dendritic cells (DCs) (Bazzi et al. 2017, Galdon et al. 2019). IMM-101 activation of immature DCs leads to the skewed maturation of activated cDC1, of which activation predominantly induces a type 1 immune response defined by the generation and maturation of IFN-γ, perforin and granzyme producing CTLs (Galdon et al., 2019), required for effective tumour cell killing. IMM-101 induced cDC1 activation also results in the generation of activated IFN-γ producing Th-1 cells NK, NKT and γδ-T cells (Fowler et al., 2014; Galdon et al., 2019), which can kill tumor cells by different mechanisms. Activated γδ-T cells are efficient antigen presenting cells (Moser et al., 2017), which may further boost anti-tumor responses. IMM-101 also activate other innate immune cells, including monocytes, which mature into M1 macrophages (Bazzi et al. 2015) that can enhance anti-tumour responses and prevent the formation of immune-suppressive M2 macrophages.

The terms “tumour,” “cancer” and “neoplasia” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumour, cancer or neoplasia to other sites, locations or regions within the subject, in which the sites, locations or regions are distinct from the primary tumour, node or cancer.

The term “non-metastatic” refers to where, relative to a primary tumour, node or cancer in a patient, there are no distant metastases or residual disease, as determined by CT, MRI or Positron emission tomography (PET) with 2-deoxy-2-[fluorine-18] fluoro-D-glucose (18F-FDG) scanning.

The terms “Programmed Death 1,” “Programmed Cell Death 1,” “Protein PD-1,” “PD-1,” and “PD1,” are used interchangeably, and include variants, isoforms, species homologs of human PD-1, and analogs having at least one common epitope with PD-1.

As used herein, “sub-therapeutic dose” means a dose of a therapeutic compound (e.g., an antibody) or duration of therapy which is lower than the usual or typical dose of the therapeutic compound or therapy of shorter duration, when administered alone for the treatment of cancer. Typical doses of known therapeutic compounds are known to those skilled in the art or can be determined through routine experimental work.

The term “therapeutically effective amount” is defined as an amount of a checkpoint inhibitor, in combination with a non-pathogenic, non-viable Mycobacterium, that preferably results in a decrease in severity of cancer disease symptoms, an increase in frequency and duration of cancer disease symptom-free periods, or a prevention of impairment or disability due to the disease affliction.

The terms “effective amount” or “pharmaceutically effective amount” refer to a sufficient amount of an agent to provide the desired biological or therapeutic result. That result can be reduction, amelioration, palliation, lessening, delaying, and/or alleviation of one or more of the signs, symptoms, or causes of a cancer or any other desired alteration of a biological system. In reference to cancer, an effective amount may comprise an amount sufficient to cause a tumour to shrink and/or to decrease the growth rate of the tumour (such as to suppress tumour growth) or to prevent or delay other unwanted cell proliferation. In some embodiments, an effective amount is an amount sufficient to delay development, or prolong survival or induce stabilisation of the cancer or tumour. Preferably, therapeutic efficacy is measured by a decrease or stabilisation of tumour size of one or more said tumours, as defined by RECIST 1.1, including stable diseases (SD), a complete response (CR) or partial response (PR) of the target tumour; and/or stable disease (SD) or complete response (CR) of one or more non-target tumours. Alternatively, therapeutic efficacy is assessed by Immune Related Response Criteria (irRC), iRECIST or irRECIST, as would be known to the skilled person. Alternatively, therapeutic efficacy of the invention described herein, is demonstrated by a pathological complete or subtotal regression, suitably measured 3, 4, or 5 weeks or more after completion of surgery or last administration of said Mycobacterium.

In some embodiments, a therapeutically effective amount is an amount sufficient to prevent or delay recurrence. A therapeutically effective amount can be administered in one or more administrations. The therapeutically effective amount of the Mycobacterium alone or in combination with surgery, preferably before and/or after said surgery, may result in one or more of the following: (i) reduce the number of cancer cells; (ii) reduce tumour size; (iii) inhibit, retard, slow to some extent and preferably stop cancer cell infiltration into peripheral organs; (iv) inhibit (i.e., slow to some extent and preferably stop) tumour metastasis; (v) inhibit tumour growth; (vi) prevent or delay occurrence and/or recurrence of tumour; and/or (vii) relieve to some extent one or more of the symptoms associated with the cancer. For example, for the treatment of tumours, a “therapeutically effective dosage” may induce tumour shrinkage by at least about 5% relative to baseline measurement, such as at least about 10%, or about 20%, or about 60% or more. The baseline measurement may be derived from untreated subjects.

A therapeutically effective amount of the Mycobacterium alone or in combination with surgery, preferably before and/or after said surgery, can decrease tumour size, or otherwise ameliorate symptoms in a subject. One of ordinary skill in the art would be able to determine such amounts based on such factors as the subject's size, the severity of the subject's symptoms, and the particular composition or route of administration selected.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells.

The term “checkpoint inhibitor” or “immunotherapy” may further include use of a cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, in practising the invention.

The term “antibody” as referred to herein includes whole antibodies and any antigen-binding fragment (i.e., “antigen-binding portion”) or single chains thereof, and includes monoclonal antibodies.

In addition to antibodies, other biological molecules may act as checkpoint inhibitors, including peptides or fusion proteins having binding affinity to the appropriate target.

By “non-viable”, it is meant that the Mycobacterium have been microbiologically inactivated through certain means of cell-killing. Methods to enable or enforce such non-viability may include heat-killing, extended freeze-drying (Tolerys SA), irradiation by gamma waves or electron beam, or subjecting the mycobacteria to chemicals such as formaldehyde. Such preparation during manufacture would mean the organism is not associated with side-effects known from delivering live or attenuated organisms.

The term “treatment” or “therapy” refers to administering an active agent with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner.

As used herein, the term “subject” or “patient” is intended to include human and non-human animals. Preferred subjects include human patients in need of enhancement of an immune response. The methods are particularly suitable for treating human patients having a disorder that can be treated by augmenting the T-cell mediated immune response. In a particular embodiment, the methods are particularly suitable for treatment of cancer cells in vivo.

As used herein, the terms “concurrent administration” or “concurrently” or “simultaneous” mean that administration occurs on the same day. The terms “sequential administration” or “sequentially” or “separate” mean that administration occurs on different days.

“Simultaneous” administration, as defined herein, includes the administration of the non-viable, Mycobacterium and one or more additional anticancer treatments or agents, within about 2 hours or about 1 hour or less of each other. Preferably “simultaneous” administration refers to wherein the non-viable, Mycobacterium and one or more additional anticancer treatments or agents are administered at the same time.

“Separate” administration, as defined herein, includes the administration of the non-viable, Mycobacterium and one or more additional anticancer treatments or agents, more than about 12 hours, or about 8 hours, or about 6 hours or about 4 hours or about 2 hours apart.

“Sequential” administration, as defined herein, includes the administration of the non-viable, Mycobacterium and one or more additional anticancer treatments or agents, each in multiple aliquots and/or doses and/or on separate occasions. The non-viable Mycobacterium may be administered to the patient after before and/or after administration of the one or more additional anticancer treatments or agents. Alternatively, the non-viable, Mycobacterium is continued to be applied to the patient after treatment with one or more additional anticancer treatments or agents.

The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, “about” means within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 20%. When particular values are provided in the application and claims, unless otherwise stated, the meaning of “about” should be assumed to be within an acceptable error range for that particular value.

M. vaccae and M. obuense have been shown to induce a complex immune response in the host. Treatment with these preparations will stimulate innate and type-1 immunity, including Th1 and macrophage activation and cytotoxic cell activity. They also independently down-regulate inappropriate Th2 responses via immunoregulatory mechanisms.

In relation to cancer, it is likely that the formation of IFN-γ producing CTLs is the most important result from IMM-101 treatment, since the observed anti-tumour effect of IMM-101 could be completely abrogated by the depletion of CD8+ T cells in a pancreas cancer model.

IMM-101's ability to activate macrophages may not only assist in the activation of DCs through the release of pro-inflammatory macrophage-derived cytokines (such as IL-12 required for skewing DCs into Type 1 immune responses), but may also be of importance for changing tumour associated immunosuppressive type 2 macrophages into tumour aggressive type 1 macrophages. This latter feature was shown for a similar heat-killed mycobacterium, M. indicus pranii.

An important feature of IMM-101 is its ability to activate and mature DCs into a sub-class of dendritic cells known as cDC1s (i.e. DCs that are required for Type 1 immune responses). It has been shown that activation of sufficient numbers of cDC1s is a prerequisite for CPIs to be effective.

Preoperative short time administration of checkpoint inhibitors in microsatellite instability (MSI) high colorectal cancers has been shown to induce high rates of pathological regression. In a recently presented small explorative phase II study (Chalabi et al. 2018, immunotherapy of cancer 29:8), six weeks of preoperative administration of the CTLA-4 antibody ipilimumab and the PD-1 antibody nivolimumab was shown to result in a complete or subtotal pathological remission, suggesting that neoadjuvant therapies could be particularly efficacious for some cancer presentations.

The present invention thus provides a protocol to use IMM-101 as a surprisingly favourable neoadjuvant treatment, or adjuvant treatment, with or without the use of other chemotherapy agents, to improve the unfavourable prognosis of patients intended to undergo tumour resection surgery or checkpoint inhibitor therapy for cancer. Such patients may include those with colorectal cancer, particularly mismatch repair-deficient (dMMR) colorectal cancers with microsatellite instability (MSI).

In a further aspect of the invention, the cancer determined as being microsatellite instability (MSI) high, as measured by a PCR-based assay and/or where the cancer is subject to IHC staining for analysis of DNA mismatch repair (MMR) protein expression

In one aspect of the present invention the non-pathogenic, non-viable Mycobacterium comprises a non-pathogenic heat-killed Mycobacterium. Examples of mycobacterial species for use in the present invention include M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M. tokaiense, M. komossense, M. aurum, M. w, M. tuberculosis, M. microti; M. africanum; M. kansasii, M. marinum; M. simiae; M. gastri; M. nonchromogenicum; M. terrae; M. triviale; M. gordonae; M. scrofulaceum; M. paraffinicum; M. intracellulare; M. avium; M. xenopi; M. ulcerans; M. diernhoferi, M. smegmatis; M. thamnopheos; M. flavescens; M. fortuitum; M. peregrinum; M. chelonei; M. paratuberculosis; M. leprae; M. lepraemurium and combinations thereof.

The non-viable, non-pathogenic Mycobacterium is preferably selected from M. vaccae, including the strain deposited under accession numbers NCTC 11659 and associated designations such as SRL172, SRP299, IMM-201, DAR-901, and the strain as deposited under ATCC 95051 (Vaccae™), M. obuense, M. paragordonae (strain 49061), M. parafortuitum, M. paratuberculosis, M. brumae, M. aurum, M. indicus pranii, M. w, M. manresensis, M. kyogaense (as deposited under DSM 107316/CECT 9546), M. phlei, M. smegmatis, M. tuberculosis Aoyama B or H37Rv, RUTI, MTBVAC, BCG, VPM1002BC, SMP-105, mifamurtide orZ-100 and combinations thereof, preferably the strain of Mycobacterium obuense deposited under the Budapest Treaty under accession number NCTC 13365.

In another most preferred embodiment, the non-pathogenic non-viable Mycobacterium is M. vaccae, including that deposited under NCTC 11569, or M. obuense, such as that deposited under NCTC 13365.In another preferred embodiment, the non-viable, non-pathogenic Mycobacterium is a rough variant.

In preferred embodiments of the invention, the non-pathogenic, non-viable Mycobacterium is the rough variant, preferably the rough variant of M. obuense.

In other embodiments of the invention, the non-pathogenic non-viable Mycobacterium is the rough variant and/or whole cell, preferably the rough strain of Mycobacterium obuense deposited under the Budapest Treaty under accession number NCTC 13365.

In another embodiment of the invention, the non-pathogenic non-viable Mycobacterium has been inactivated by heat such as autoclaving, extended freeze drying, chemical exposure such as formaldehyde, or irradiation such as gamma irradiation or e-beam.

In another embodiment of the invention, the non-pathogenic non-viable Mycobacterium does not include BCG in live, attenuated form.

In a further embodiment, the non-pathogenic non-viable Mycobacterium is the rough variant and/or a presented as a fraction, fragment, sub-cellular component, lysate, homogenate, sonicate, or substantially in whole cell form.

In preferred embodiments of the invention, the non-pathogenic, non-viable Mycobacterium, suitably Mycobacterium obuense, is in a substantially whole cell form, such as where more than 50% or more of the mycobacteria in suspension are greater than 1 to 10 microns in diameter, as measured by laser diffraction (e.g. D50 value or mean particle size), or is in a form which has not been exposed to high pressure processing or other conditions to induce substantial cell lysis.

As would be understood by the skilled person, rough variants of M. obuense, for example, would lack cell surface-associated glycopeptidolipids (GPL) resulting in a characterised rough morphology with non-motile and non-biofilm-forming properties, as described in Roux et al. 2016, Open Biol 6: 160185. The amount of Mycobacterium administered to the patient in the present invention would be sufficient to elicit a protective immune response in the patient such that the patient's immune system would be able to mount an effective immune response In another most preferred embodiment, the cancer is determined as being colorectal cancer (CRC), wherein said cancer or tumour is found in the ascending, descending and/or descending colon, and/or the rectum.

In some embodiments, the cancer is determined as being colon cancer, optionally metastatic.

In other embodiments, the cancer is determined as being rectal cancer, optionally metastatic.

The amount of Mycobacterium administered to the patient is sufficient to elicit an immune response in the patient such that the patient's immune system is able to mount an effective immune response to the cancer or tumour. In certain embodiments of the invention, there is provided a containment means comprising the effective amount of Mycobacterium for use in the present invention, which typically may be from 10³ to 10¹¹ organisms, preferably from 10⁴ to 10¹⁰ organisms, more preferably from 10⁶ to 10¹⁰ organisms, and even more preferably from 10⁶ to 10⁹ organisms. Most preferably the amount of Mycobacterium for use in the present invention is from 10⁷ to 10⁹ cells or organisms. Typically, the composition according to the present invention may be administered at a dose of from 10⁸ to 10⁹ cells for human and animal use. Alternatively, the dose is from 0.0001 mg to 5 mg or 0.001 mg to 5 mg organisms, preferably 0.01 mg to 2 mg or 0.1 mg to 2 mg organisms, such as where the dose is approximately 0.5 mg or 1 mg or 0.5 mg or 1 mg organisms. The dose may be prepared as either a suspension or dry preparation.

In certain embodiments of the invention, the amount of non-pathogenic non-viable Mycobacterium administered is between 0.0001 mg and 1 mg per unit dose, optionally wherein the unit dose is administered on two or more separate occasions separated by at least 7 days or more, such as administration on each of day 0, day 14 (+/−1, 3 or 5 days or more), and optionally day 30 (+/−5, 7 or 10 days or more) or day 45 (+/−7, 10 or 14 days or more).

In some embodiments, the amount of non-pathogenic non-viable Mycobacterium administered may be from 0.0001 mg to 1 mg per dose wherein the dose is administered 1, 2, 3, 4, 5, 6, 10 or 20 or more times over a number of days, weeks, or months, suitably wherein the Mycobacterium is M. obuense.

In other embodiments of the invention, the amount of non-pathogenic non-viable Mycobacterium administered may be from 0.0001 mg to 1 mg per dose, wherein the dose initially comprises one injection of 1 mg into one deltoid, or two injections of 0.5 mg in each deltoid, or two injections of 1.0 mg in each deltoid, followed by a second dose of either 0.5 or 1.0 mg 7 or 14 days or more later.

In certain embodiments of the invention, the amount of non-pathogenic non-viable Mycobacterium administered is between 0.0001 mg and 1 mg per unit dose, such as between about 0.5 and 1 mg, wherein the unit dose is administered on two or more separate occasions separated by at least 7 days or more, prior to surgery, such as administration on each of day −35, day −21 and day −5 relative to (i.e. prior to) surgery, optionally where the −35 day dose is 1 mg, the −21 day dose is 0.5 mg and the −5 day dose is 0.5 mg. The checkpoint therapy, such as an anti-PD-L1 mab, suitably atezolizumab, pembrolizumab or cemiplimab, may be administered on the same day or in between the unit dose of said Mycobacterium, such as day −28 and day −7, relative to (i.e. prior to) said surgery.

In a further embodiment of the invention, the amount of non-pathogenic non-viable Mycobacterium administered is between 0.0001 mg and 1 mg per unit dose, such as between about 0.5 and 1 mg, wherein the unit dose is administered on two or more separate occasions separated by at least 7 days or more, after surgery, such as administration on each of day 21, day 35, day 49 et seq., following said surgery, optionally where the day 21 dose is 1 mg, the 35 day dose is 0.5 mg and the day 49 dose onward, is 0.5 mg. The checkpoint therapy, such as an anti-PD-L1 mab, suitably atezolizumab, pembrolizumab or cemiplimab, may be administered on the same day or in between the unit dose of said Mycobacterium, such as day 28, day 42, day 56, et seq., following said surgery.

In yet another embodiment of the invention, the amount of non-pathogenic non-viable Mycobacterium administered is between 0.0001 mg and 1 mg per unit dose, such as between about 0.5 and 1 mg, wherein the unit dose is administered on two or more separate occasions separated by at least 7 days or more, prior to surgery, such as administration on each of day −35, day −21 and day −5 relative to (i.e. prior to) surgery, optionally where the −35 day dose is 1 mg, the −21 day dose is 0.5 mg and the −5 day dose is 0.5 mg. The checkpoint therapy such as an anti-PD-L1 mab, suitably atezolizumab, pembrolizumab or cemiplimab, may be administered on the same day or in between the unit dose of said Mycobacterium, such as day −28 and day −7, relative to (i.e. prior to) said surgery, wherein a unit dose is administered on two or more separate occasions separated by at least 7 days or more, after surgery, such as administration on each of day 21, day 35, day 49 et seq., following said surgery, optionally where the day 21 dose is 1 mg, the 35 day dose is 0.5 mg and the day 49 dose onward, is 0.5 mg.

The checkpoint therapy such as an anti-PD-L1 mab, suitably atezolizumab, pembrolizumab or cemiplimab, may be administered on the same day or in between the unit dose of said Mycobacterium, such as day 28, day 42, day 56, et seq., following said surgery. The present invention may be used to treat neoplastic disease, such as solid or non-solid cancers.

As used herein, “treatment” encompasses the prevention, reduction, control and/or inhibition of a neoplastic disease, including the regression or stabilization of a primary tumour and/or the regression or stabilization of one or metastases, or the prevention or inhibition of one or more metastases or micrometastases.

Neoplasia, tumours and cancers include benign, malignant, metastatic and non-metastatic types, and include any stage (I, II, III, IV or V) or grade (G1, G2, G3, etc.) of neoplasia, tumour, or cancer, or a neoplasia, tumour, cancer or metastasis that is progressing, worsening, stabilized or in remission. Preferably, the cancer at the onset of practising the invention is clinically defined as being Stage I, Stage II or Stage II. Cancers that may be treated according to the invention include but are not limited to: bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to the following: neoplasm, malignant carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumour, malignant bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant ovarian stromal tumour, malignant thecoma, malignant granulosa cell tumour, malignant androblastoma, malignant Sertoli cell carcinoma; Leydig cell tumour, malignant lipid cell tumour, malignant paraganglioma, malignant extra-mammary paraganglioma, malignant pheochromocytoma; glomangiosarcoma; malignant melanoma; amelanotic melanoma; superficial spreading melanoma; malignant melanoma in giant pigmented nevus; metastatic melanoma, Lentigo Maligna, Lentigo Maligna Melanoma, cutaneous squamous cell carcinoma, Nodular Melanoma, Acral Lentiginous Melanoma, desmoplastic Melanoma, epithelioid cell melanoma; blue nevus, malignant sarcoma; fibrosarcoma; fibrous histiocytoma, malignant myxosarcoma; liposarcoma; leiomyosarcoma; rhabdomyosarcoma; embryonal rhabdomyosarcoma; alveolar rhabdomyosarcoma; undifferentiated pleomorphic sarcoma; stromal sarcoma; mixed tumour; Mullerian mixed tumour; nephroblastoma; hepatoblastoma; carcinosarcoma; mesenchymoma, malignant Brenner tumour, malignant phyllodes tumour, malignant synovial sarcoma; mesothelioma, malignant dysgerminoma; embryonal carcinoma; teratoma, malignant; struma ovarii, malignant; choriocarcinoma; mesonephroma, malignant hemangiosarcoma; hemangioendothelioma, malignant Kaposi's sarcoma; hemangiopericytoma, malignant lymphangiosarcoma; osteosarcoma; juxtacortical osteosarcoma; chondrosarcoma; chondroblastoma, malignant mesenchymal chondrosarcoma; giant cell tumour of bone; Ewing's sarcoma; odontogenic tumour, malignant ameloblastic odontosarcoma; ameloblastoma, malignant ameloblastic fibrosarcoma; pinealoma, malignant chordoma; glioma, malignant ependymoma; astrocytoma; protoplasmic astrocytoma; fibrillary astrocytoma; astroblastoma; glioblastoma multiforme; oligodendroglioma; oligodendroblastoma; primitive neuroectodermal; cerebellar sarcoma; ganglioneuroblastoma; neuroblastoma; retinoblastoma; olfactory neurogenic tumour; meningioma, malignant neurofibrosarcoma; neurilemmoma, malignant granular cell tumour, malignant lymphoma; Hodgkin's disease; Hodgkin's paragranuloma; malignant lymphoma, small lymphocytic malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Preferably, the cancer is selected from bladder cancer (including non-muscle invasive bladder cancer), prostate cancer, liver cancer, renal cancer, lung cancer, breast cancer, colorectal cancer, colon cancer, rectal cancer, pancreatic cancer, brain cancer (including glioblastoma), hepatocellular cancer, lymphoma, leukaemia, gastric cancer, cervical cancer, ovarian cancer, thyroid cancer, melanoma, head and neck cancer, skin cancer and soft tissue sarcoma and/or other forms of carcinoma.

More preferably, the cancer is pancreatic, colorectal, brain, prostate, skin, soft-tissue sarcoma, osteosarcoma, lung or ovarian cancer, including pancreatic ductal adenocarcinoma (PDAC).

In some embodiments of the invention, the early-stage cancer is defined by a staging criteria, such as the TNM classification system. The TNM classification system describes the stage of a cancer, which originates from a solid tumour, using alphanumeric codes, in which: T describes aspects of the original primary tumour, including its size and growth; N describes nearby or regional lymph nodes that may be involved; and M describes the level of spread, or metastasis, of the tumour. A classification of TX tells the skilled person that the tumour cannot be measured; TO describes no evidence of a primary tumour; and T1, T2, T3 and T4 consecutively describe the increasing tumour size and/or amount of growth or spread into nearby structures, wherein the higher the T number, the larger the tumour and/or tumour growth and spread. A classification of NX similarly means that the nearby lymph nodes could not be evaluated; N0 means nearby lymph nodes do not contain cancer; and N1, N2 and N3 each describe the increasing size, location, and/or number of nearby lymph nodes affected. Lastly, M0 means that no distant cancer spread was found, or that the tumour is not metastatic, whereas M1 means that the cancer has spread and has metastasised.

In a preferred embodiment, the early-stage cancer is defined in particular by a T of T1-T4, and optionally an M of M0, whilst N can be any classification, such as N0, N1 or N2. Alternatively, said cancer is clinically defined as being Stage I, Stage II or Stage Ill. In other words, the cancer is defined by a recorded size and growth of any size, and further optionally the tumour is non-metastatic.

In a further preferred embodiment or method of the invention, the patient does not present with metastases in any organs distant to the primary tumour but may present with metastases in one or more lymph nodes near to the primary neoplasia or tumour, and/or may present with metastases in one or more nearby organs. Alternatively, said patient does not present with any macroscopic residual disease following tumour resection surgery, as demonstrated by an R2 resection status.

In a further preferred embodiment or method of the invention, the patient is clinically defined as non-metastatic.

In a further preferred embodiment or method of the invention, the neoplasia, tumour or cancer is associated with a sarcoma, preferably a soft tissue or non-soft tissue sarcoma. Particularly preferred non-soft tissue sarcomas include bone sarcomas (osteosarcoma, Ewing's sarcoma) and chondrosarcoma. Particularly preferred sarcomas include pleomorphic undifferentiated sarcoma (UPS), angiosarcoma, leiomyosarcoma, non-uterine leiomyosarcoma, dedifferentiated liposarcoma (DDL), synovial sarcoma, rhabdomyosarcoma, epithelioid sarcoma, myxoid liposarcoma, alveolar soft part sarcoma, parachordoma/myoepithelioma, pleomorphic liposarcoma, extraskeletal myxoid chondrosarcoma, or malignant peripheral nerve sheath tumors. The patient may be less than 50 years of age, or less than 20 to 30 years of age, or a teenager or adolescent (<16 years of age), or a child (0 to 14 years of age). Optionally, the one or more sarcoma tumours demonstrate increased staining/expression of PD-L1 or PD-1. Optionally, the non-viable Mycobacterium and/or checkpoint inhibitor and/or co-stimulatory binding agent is administered via intratumoral, peritumoral, perilesional or intralesional administration.

The present invention relates to patients with cancer that have undergone, or are intended to undergo, tumour resection surgery or checkpoint inhibitor therapy. The term “intended to undergo” may refer to patients that have been advised and/or managed such that they will receive said tumour resection surgery or checkpoint inhibitor therapy as part of their protocol of health management and/or care plan. In some embodiments, the immunomodulator for use in the present invention may be used in the treatment, reduction, inhibition or control of early-stage cancer in a patient intended to undergo tumour resection surgery or checkpoint inhibitor therapy up to one year in the future. In other embodiments, the patient may be intended to undergo tumour resection surgery or checkpoint inhibitor therapy up to six months in the future. In preferred embodiments, the patient may be intended to undergo tumour resection surgery or checkpoint inhibitor therapy up to six weeks in the future.

Wherein the present invention relates to subjects that have undergone, or are intended to undergo, checkpoint inhibitor therapy, said checkpoint inhibition therapy may comprise administration of one or more blocking agents, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof.

In an embodiment of the invention, the checkpoint inhibition therapy may comprise administration of a sub-therapeutic amount and/or duration of said one or more blocking agents.

In an embodiment of the invention, the one or more blocking agents are selected from ipilimumab, nivolumab, pembrolizumab, azetolizumab, BI 754091(anti-PD-1), bavituximab (an IgG3 mab against PS), bintrafusp alfa, dostarlimab, durvalumab, tremelimumab, spartalizumab, avelumab, sintilimab, toripalimab, prolgolimab, tislelizumab, camrelizumab, MGA012, MGD013 (now known as tebotelimab), MGD019, nimotuzumab, enoblituzumab, MGD009, MG0018, MED10680, miptenalimab (BI 754111, an anti-LAG-3), PDR001, FAZ053, TSR022, MBG453, relatlimab (BMS986016), LAG525 (IMP701), IMP321 (Eftilagimod alpha), REGN2810 (cemiplimab), REGN3767, pexidartinib (PLX3397), LY3022855, FPA008, BLZ945, GDC0919, epacadostat, emactuzumab (RG1755 targeting CSF-1R), FPA150, indoximid, BMS986205, CPI-444, MEDI9447, PBF509, FS118 (bispecificfor LAG-3 and PD-L1), lirilumab, Sym023, TSR-022, A2Ar inhibitors (e.g. EOS100850, AB928), NKG2A inhibitors such as monalizumab, and combinations thereof.

In an embodiment of the invention, the one or more blocking agents are preferably ipilimumab and/or nivolumab.

In an embodiment of the invention, the co-stimulatory checkpoint therapy comprises administration of one or more binding agents selected from utomilumab, urelumab, MOXR0916. PF04518600, MED10562, GSK3174988, MED16469. R07009789, CP870893, BMS986156, GWN323, JTX-2011, varlilumab, MK-4166, NKT-214 and combinations thereof.

In an embodiment of the invention, the one or more blocking agents or checkpoint inhibitors is most preferably atezolizumab.

In an embodiment of the invention, the checkpoint inhibitor therapy comprises administration of one or more blocking agents, selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1R, CD94/NKG2A, TDO, TNFR, DcR3 and combinations thereof, in combination with a non-viable, Mycobacterium, to reduce or inhibit metastasis of a primary tumour or cancer to other sites, or the formation or establishment of metastatic tumours or cancers at other sites distal from the primary tumour or cancer thereby inhibiting or reducing tumour or cancer relapse or tumour or cancer progression.

In an embodiment of the invention, there is provided a non-pathogenic, non-viable Mycobacterium for use in the adjuvant, neoadjuvant or peri-adjuvant treatment, reduction, inhibition or control of one or more tumours in a subject that has undergone, or is intended to undergo, tumour resection surgery or checkpoint inhibitor therapy, with the potential to elicit potent and durable immune responses with enhanced therapeutic benefit compared to either surgery or therapy alone, preferably as measured by a decrease or stabilisation of tumour size of one or more said tumours, as defined by RECIST 1.1, or iRRC, or iRECIST, or irrRECIST, including stable diseases (SD), a complete response (CR) or partial response (PR) of the target tumour; and/or stable disease (SD) or complete response (CR) of one or more non-target tumours.

In some embodiments, the checkpoint inhibitor therapy of the invention may be co-stimulatory checkpoint therapy, wherein said co-stimulatory checkpoint therapy comprises administration of one or more binding agents selected from a cell, protein, peptide, antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CD27, CD28, CD40, CD122, CD137, OX40, GITR, ICOS and combinations thereof.

In an embodiment of the invention, the co-stimulatory checkpoint therapy comprises administration of one or more binding agents selected from utomilumab, urelumab, MOXR0916. PF04518600, MED10562, GSK3174988, MED16469. R07009789, CP870893, BMS986156, GWN323, JTX-2011, varlilumab, MK-4166, NKT-214 and combinations thereof.

In some further embodiments, the checkpoint inhibition therapy may comprise administration of two or more blocking agents, wherein said two or more blocking agents are directed against any one of the following combinations of receptor targets: CTLA-4 and PD-1, CTLA-4 and PD-L1, PD-1 and LAG-3, or PD-1 and PD-L1.

Suitable specific combinations include: Durvalumab+tremelimumab, Nivolumab+ipilimumab, Pembrolizumab+ipilimumab, MEDI0680+durvalumab, PDR001+FAZ053, Nivolumab+ TSR022, PDR001+MBG453, Nivolumab+BMS 986016, PDR001+LAG525, Pembrolizumab+IMP321, REGN2810 (cemiplimab)+REGN3767, and other suitable combinations.

In some embodiments, the checkpoint inhibitor therapy may further comprise co-stimulatory checkpoint therapy, directed against any one of the following combinations: CTLA-4 and CD40, CTLA-4 and OX40, CTLA-4 and IDO, OX-40 and PD-L1, PD-1 and OX-40, CD27 and PD-L1, PD-1 and CD137, PD-L1 and CD137, OX-40 and CD137, CTLA-4 and IDO, PD-1 and IDO, PD-L1 and IDO, PD! And A2AR, PD-L1 and A2AR, PD1 and GITR, PD-L1 and GITR, PD1 and ICOS, PD-L1 and ICOS, PD1 and CD27, PD-L1 and CD27, PD1 and CD122, PD-L1 and CD122, PD1 and CSF1R, PD-L1 and CSF1R, and other such suitable combinations.

Suitable specific combinations include: Avelumab+utomilumab, Nivolumab+urelumab, Pembrolizumab+utomilumab, Atezolimumab+MOXR0916±bevacizumab, Avelumab+PF-04518600, Durvalumab+MED10562, Pembrolizumab+GSK3174998, Tremelimumab+durvalumab+MEDI6469, Tremelimumab+MED10562, Utomilumab+PF-04518600, Atezolimumab+R07009789, Tremelimumab+CP870893, Nivolumab+BMS986156, PDR001+GWN323, Nivolumab+JTX-2011, Atezolizumab+GDC0919, Ipilimumab+epacadostat, Ipilimumab+indoximid, Nivolumab+BMS986205, Pembrolizumab+epacadostat, Atezolizumab+CPI-444, Durvalumab+MEDI9447, PDR001+PBF509, Nivolumab+varlilumab, Atezolizumab+varlilumab, Nivolumab+NKTR-214, Durvalumab+Pexidartinib (PLX3397), Durvalumab+LY3022855, Nivolumab+FPA008, Pembrolizumab+Pexidartinib, PDR001+BLZ945, Tremelimumab+LY3022855.

In a further embodiment, the checkpoint inhibitor therapy comprises administration of a blocking agent, wherein said blocking agent is an antibody selected from the group consisting of: AMP-224 (Amplimmune, Inc), BMS-986016 (relatlimab) or MGA-271, and combinations thereof.

In certain embodiments, the co-stimulatory checkpoint therapy upregulates the cellular immune system, wherein said co-stimulatory checkpoint therapy comprises administration of a binding agent, selected from a cell, protein, peptide, antibody or antigen binding fragment thereof, directed against CD27, OX40, GITR, or CD137, and combinations thereof, such as CD137 agonists including without limitation BMS-663513 (urelumab, an anti-CD137 humanized monoclonal antibody agonist, Bristol-Myers Squibb); agonists to CD40, such as CP-870,893 (a-CD40 humanized monoclonal antibody, Pfizer); OX40 (CD 134) agonists (e.g. anti-OX40 humanized monoclonal antibodies, AgonOx and those described in U.S. Pat. No. 7,959,925), and Astra Zeneca's MED10562, a humanised OX40 agonist; MEDI6469, murine OX4 agonist; and MEDI6383, an OX40 agonist; or agonists to CD27 such as CDX-1127 (a-CD27 humanized monoclonal antibody, Celldex). Suitable anti-GITR antibodies include TRX518 (Tolerx), MK-1248 (Merck), CK-302 and suitable anti-4-11BB antibodies for use in the invention include PF-5082566 (Pfizer).

A most preferred TLR3 agonist for use in the invention is rintatolimod (Ampligen).

In an embodiment of the invention, the checkpoint inhibition therapy and/or the co-stimulatory checkpoint therapy act synergistically with the Mycobacterium.

In some embodiments of the invention, the treatment, reduction, inhibition or control of the early-stage cancer further comprises administration of one or more additional anticancer treatments or agents.

The one or more additional anticancer treatments or agents may be selected from: adoptive cell therapy, surgical therapy, chemotherapy, radiation therapy, hormonal therapy, checkpoint inhibitor therapy, small molecule therapy such as metformin, receptor kinase inhibitor therapy such as tyrosine kinase inhibitor therapy, hyperthermia treatment, phototherapy, radiofrequency ablation therapy (RFA), anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonists including TLR2, 3, 4, 7, 8 or 9 agonists, or TLR 5 agonists such as MRx0518 (4D Pharma), STING agonists (including MIW815 and SYNB1891), mifamurtide and cancer vaccines such as GVAX or CIMAvax, and combinations thereof.

In another embodiment of the invention, the one or more additional anticancer treatments results in immunogenic cell death therapy, as described in WO2013/07998. This therapy results in the induction of tumour immunogenic cell death, including apoptosis (type 1), autophagy (type 2) and necrosis (type 3), whereupon there is a release of tumour antigens that are able to both induce immune responses, including activation of cytotoxic CD8+ T cells and NK cells and to act as targets, including rendering antigens accessible to Dendritic Cells. The immunogenic cell death therapy may be carried out at sub-optimal levels, i.e. non-curative therapy such that it is not intended to fully remove or eradicate the tumour, but nevertheless results in some tumour cells or tissue becoming necrotic. The skilled person will appreciate the extent of therapy required in order to achieve this, depending on the technique used, age of the patient, status of the disease and particularly size and location of tumour or metastases Particularly preferred treatments include: microwave irradiation, targeted radiotherapy such as stereotactic ablative radiation (SABR), embolisation, cryotherapy, ultrasound, high intensity focused ultrasound, cyberknife, hyperthermia, radiofrequency ablation, cryoablation, electrotome heating, hot water injection, alcohol injection, embolization, radiation exposure, photodynamic therapy, laser beam irradiation, and combinations thereof.

In a further embodiment of the invention, the TLR agonists include MRx0518 (4D Pharma), mifamurtide (Mepact), Krestin (PSK), IMO-2125 (tilsotolimod), CMP-001, MGN-1703 (lefitolimod), entolimod, rintatolimod (Ampligen), SD-101, GS-9620, imiquimod, resiquimod, MED14736, poly I:C, CPG7909, DSP-0509, VTX-2337 (motolimod), MED19197, NKTR-262, G100 or PF-3512676 and combinations thereof.

In a further embodiment of the invention, the chemotherapy comprises administration of one or more agents selected from: cyclophosphamide, methotrexate, 5-fluorouracil, doxorubicin, mustine, vincristine, procarbazine, prednisolone, bleomycin, vinblastine, dacarbazine, etoposide, cisplatin, epirubicin, capecitabine, leucovorin, folinic acid, carboplatin, oxaliplatin, gemcitabine, FOLFIRINOX, modified FOLIFIRINOX, FOLFOX, paclitaxel, pemetrexed, irinotecan temozolomide and combinations thereof.

In a further embodiment of the invention, the therapy comprises atezolizumab in combination with folinic acid, fluorouracil, and oxaliplatin (FOLFOX) and bevacizumab, suitably as an adjuvant, neoadjuvant or peri-adjuvant therapy in patients with stage III dMMR and/or MSI-High colorectal cancer (CRC). Alternatively, patients may receive atezolizumab plus FOLFOX and bevacizumab, for 6 months, with or without a Mycobacterium, followed by azetolizumab with or without bevacizumab and/or with or without a Mycobacterium, for a further 6 months or more.

In a further embodiment of the invention, the therapy comprises atezolizumab in combination with folinic acid, fluorouracil, and oxaliplatin (FOLFOX), suitably as an adjuvant, neoadjuvant or peri-adjuvant therapy in patients with stage III dMMR and/or MSI-High CRC. Alternatively, patients may receive atezolizumab plus FOLFOX for 6 months, with or without a Mycobacterium.

In a further embodiment of the invention, the combination is suitable for treatment of pMMR-MSI-Low CRC tumours, i.e. mismatch repair proficient and microsatellite instability low tumours, such as combinations with or without FOLFOX and checkpoint inhibitors such as pemobrolizumab and/or atezolizumab, or durvalumab and/or tremelimumab, in combination with a Mycobacterium. Other combinations include atezolizumab plus Bevacizumab together with a Mycobacterium.

In a further embodiment of the invention, the combination is suitable for treatment of pMMR-MSI-Low CRC tumours, wherein the non-pathogenic, non-viable Mycobacterium is administered in combination with one or more of the following combinations: RFA and/or EBRT and/or pembrolizumab; RFA and/or durvalumab and/or tremelimumab; atezolizumab and/or bevacizumab and/or FOLFOX; atezolizumab and cobimetinib; nivolumab+ipilimumab+cobimetinib; atezolizumab+cobimetinib+bevacizumab; atezolizumab+CEA-BTC antibody; Pembrolizumab+indoximod; nivolumab+epacadostat; durvalumab+pexidartinib.

In a further embodiment of the invention, the combination comprises either radiofrequency ablation (RFA) or external beam radiation therapy (EBRT) in patients with CRC, together with checkpoint inhibitors such as pemobrolizumab and/or atezolizumab, or durvalumab and/or tremelimumab, in combination with a Mycobacterium.

In a further embodiment of the invention, the adjuvant, neoadjuvant or peri-adjuvant therapy disclosed herein is applied to patients with stage III dMMR and/or MSI-High colorectal cancer, or colon cancer or rectal cancer, wherein said patient presents with an ECOG Performance Status of 0 (PS0), 1 (PS1) or PS1-, or 2 (PS2).

In a further embodiment of the invention, the adjuvant, neoadjuvant or peri-adjuvant therapy disclosed herein, is applied to patients who present with an ECOG Performance Status of 0 (PS0), 1 (PS1) or PS1- or 2 (PS2) and wherein said Performance Status value is the same or improved when assessed post-surgery or at the end of therapy.

In a further embodiment of the invention, the adjuvant, neoadjuvant or peri-adjuvant therapy disclosed herein is applied to patients with stage III pMMR/MSI-Low colorectal cancer, or colon cancer or rectal cancer i.e. mismatch repair proficient and microsatellite instability low tumours in patients with colorectal cancer, or colon cancer or rectal cancer, wherein said patient presents with an ECOG Performance Status of 0 (PS0), 1 (PS1) or PS1- or 2 (PS2).

In some preferred embodiments, the one or more additional anticancer treatments or agents may include checkpoint inhibitor agents. Such checkpoint inhibitors may be selected from ipilimumab, nivolumab, pembrolizumab, atezolizumab, bintrafusp alfa, dostarlimab, durvalumab, tremelimumab, spartalizumab, avelumab, sintilimab, toripalimab, prolgolimab, tislelizumab, camrelizumab, MGA012, MGD013, MGD019, enoblituzumab, MGD009, MGC018, MEDI0680, PDR001, FAZ053, TSR022, MBG453, relatlimab (BMS986016), LAG525, IMP321, REGN2810 (cemiplimab), REGN3767, pexidartinib, LY3022855, FPA008, BLZ945, GDC0919, epacadostat, indoximid, BMS986205, CPI-444, MEDI9447, PBF509, and combinations thereof.

In a further embodiment of the invention, the one or more additional anticancer treatments or agents, and/or Mycobacteria, are administered intratumorally, intraarterially, intravenously, intravascularly, intrapleurally, intraperitoneally, intratracheally, intranasally, intranodally, pulmonarily, intrathecally, intramuscularly, endoscopically, intralesionally, perilesionally, peritumorally, percutaneously, subcutaneously, regionally, stereotactically, orally or by direct injection or perfusion.

The appropriate ranges of dosages and administration protocols appropriate for each one or more additional anticancer treatments or agents would be appreciated within the art. By way of an example, an appropriate dosage protocol for atezolizumab may be 1200 mg, every 3 weeks. In some embodiments, atezolizumab may be administered intravenously, at a dose of 1200 mg every 3 weeks, for up to 12 months or longer. In a further embodiment, the atezolizumab may be administered intravenously every 3 weeks for 6 to 8 weeks prior to tumour resection surgery or further checkpoint inhibitor therapy.

In some embodiments, atezolizumab may be administered intravenously, at a dose of 1200 mg every 3 weeks (+/−3 days), 7 days and 28 days prior to tumour resection, with administration of the Mycobacterium on the same or different day, such as 35, days and/or 21 days and/or 5 days prior to resection. Alternatively, atezolizumab may be administered intravenously, at a dose of 840 mg every 2 weeks (+/−3 days), starting 28 days subsequent to tumour resection, with administration of the Mycobacterium on the same or different day, such as day 21, day 35 day 42 et seq, following said tumour resection.

In a further embodiment, atezolizumab may be administered intravenously, at a dose of 1200 mg every 3 weeks (+/−3 days), 7 days and 28 days prior to tumour resection, with administration of the Mycobacterium on the same or different day, such as 35 days, 21 days and 5 days prior to resection. Further wherein, atezolizumab is to be administered intravenously, at a dose of 840 mg every 2 weeks (+/−3 days), starting 28 days subsequent to tumour resection, with administration of the Mycobacterium on the same or different day, such as day 21, day 35 day 42 et seq, following said tumour resection.

In preferred embodiments of the invention, said tumour resection results in a tumour margin as defined according to the AJCC 8^{th} Edition, such as an R0 resection margin, where no cancer cells are seen microscopically at the primary tumour site; or R1, where cancer cells are present microscopically at the primary tumour site; or, R2, where macroscopic residual tumour is found at the primary cancer site and/or regional lymph nodes.

In a further embodiment, a combination or Mycobacterium according to the invention is administered within 1, 2, 5, 10, 20, 30, 40, 50, 60 or 70 days after tumour resection, wherein said tumour resection is suitably an R0, R1 or R2 resection, preferably R0 and no later than 70 days after. Alternatively, a combination or Mycobacterium according to the invention described herein, is administered in one or more doses within 1, 2, 5, 10, 20, 30, 40, 50, 60 or 70 days before a planned R0, R1 or R2 tumour resection surgery, most preferably an R0 resection. A further alternative embodiment, provides a combination or Mycobacterium according to the invention described herein, administered in one or more doses within 1, 2, 5, 10, 20, 30, 40, 50, 60 or 70 days before a planned R0, R1 or R2 tumour resection surgery, most preferably an R0 resection, followed by application of a combination or Mycobacterium according to the invention, where either or both are administered within 1, 2, 5, 10, 20, 30, 40, 50, 60 or 70 days after said tumour resection.

In a further embodiment, within 70 days post tumour resection, the Mycobacterium may be initially administered intradermally, at an initial dose of 1.0 mg at 14±2 days prior to the administration of the first infusion of atezolizumab, followed by administration of atezolizumab intravenously at a dose of 840 mg every 2 weeks, for a period of 12 months following said tumour resection, combined with administration of the Mycobacterium at a dose of 0.5 mg every 2 weeks for one month, followed by a dose of 0.5 mg Mycobacterium every 4 weeks for 11 months, on the same or different day as said atezolizumab infusion, preferably wherein said Mycobacterium is M. obuense NCTC 13365.

In a further embodiment, atezolizumab may be administered intravenously at a dose of 1200 mg 28 days and 7 days prior to tumour resection, with administration of the Mycobacterium, administered intradermally, at a dose of 1.0 mg 35 days prior to tumour resection, followed by administration of the Mycobacterium, at a dose of 0.5 mg 21 days and 5 days prior to tumour resection, preferably wherein said Mycobacterium is M. obuense NCTC 13365.

In a further embodiment, following surgery (adjuvant setting), atezolizumab may be administered intravenously at a dose of 840 mg every 2 weeks for 12 months or more following said tumour resection, with administration of the Mycobacterium administered intradermally, initially at a dose of 1.0 mg following said tumour resection, followed by administration of the Mycobacterium at a dose of 0.5 mg every 2 weeks for one month, followed by a dose of 0.5 mg Mycobacterium every 4 weeks for 11 months, on the same or different day as said atezolizumab infusion, preferably wherein said Mycobacterium is M. obuense NCTC 13365.

In a further embodiment, the invention comprises a peri-adjuvant regimen, wherein atezolizumab may be administered intravenously at a dose of 1200 mg 28 days and 7 days, prior to tumour resection, with administration of the Mycobacterium, administered intradermally, at a dose of 1.0 mg 35 days prior to tumour resection, followed by administration of the Mycobacterium, at a dose of 0.5 mg 21 days and 5 days prior to tumour resection (neoadjuvant setting), and wherein following said surgery (adjuvant setting), atezolizumab may be administered intravenously at a dose of 840 mg every 2 weeks for 12 months or more following said tumour resection, with administration of the Mycobacterium administered intradermally, initially at a dose of 1.0 mg following said tumour resection, followed by administration of the Mycobacterium at a dose of 0.5 mg every 2 weeks for one month, followed by a dose of 0.5 mg Mycobacterium every 4 weeks for 11 months, on the same or different day as said atezolizumab infusion, preferably wherein said Mycobacterium is M. obuense NCTC 13365.

In a further embodiment, the invention comprises a neo-adjuvant, adjuvant or peri-adjuvant regimen in a human subject, wherein said non-viable, non-pathogenic Mycobacterium and one or more additional anticancer treatments or agents are administered, optionally at the same or different time, via the same or different route of administration, and where the human subject demonstrates a pathological complete or particle/subtotal response or tumour regression at 5 weeks or later post-surgery or end of therapy, and/or an increased disease-free survival (DFS) or Overall Survival (OS) at 1, 2, 3 or 5 years or later, post-surgery or end of therapy, and/or an improved quality of life (assessed by EORTC QLQ-C30 and PRO-CTCAE questionnaires), and/or no detectable ctDNA at 12 months or later, post-surgery or end of therapy, preferably wherein said Mycobacterium is M. obuense NCTC 13365.

Circulating tumor DNA (“ctDNA”) is found in the bloodstream and refers to DNA that comes from cancerous cells and tumours. Measurement of ctDNA has emerged as a promising blood-based biomarker for monitoring disease status of patients with advanced cancers. The presence of ctDNA in the blood is a result of biological processes, namely tumour cell apoptosis and/or necrosis, and can be used to monitor different cancers by targeting cancer-specific mutation.

In a further embodiment of the invention, there is provided an immunomodulator for use in the adjuvant, neoadjuvant or peri-adjuvant treatment, reduction, inhibition or control of cancer in a patient that has undergone, or is intended to undergo, tumour resection surgery or checkpoint inhibitor therapy, optionally further comprising the administration of one or more additional anticancer agents or treatments, optionally wherein said patient does not present with metastases in any organs distant to the primary tumour, wherein said immunomodulator comprises a non-pathogenic non-viable Mycobacterium which mediates any combination of at least one of the following immunostimulatory effects on immunity, preferably wherein said Mycobacterium is M. obuense NCTC 13365: (i) increasing immune response, (ii) increasing T cell activation, (iii) increasing cytotoxic T cell activity, (iv) increasing NK cell activity, (v) increasing Th17 activity, (vi) alleviating T-cell suppression, (vii) increasing pro-inflammatory cytokine secretion, (viii) increasing IL-2 secretion, (ix) increasing interferon-y production by T-cells, (x) increasing Th1 response, (xi) decreasing Th2 response, (xii) decreasing or eliminating at least one of regulatory T cells (Tregs), myeloid derived suppressor cells (MDSCs), iMCs, mesenchymal stromal cells, TIE2-expressing monocytes, (xiii) reducing regulatory cell activity and/or the activity of one or more of myeloid derived suppressor cells (MDSCs), iMCs, mesenchymal stromal cells, TIE2-expressing monocytes, (xiv) decreasing or eliminating M2 macrophages, (xv) reducing M2 macrophage pro-tumorigenic activity, (xvi) decreases or eliminates N2 neutrophils, (xvii) reduces N2 neutrophils pro-tumorigenic activity, (xviii) reducing inhibition of T cell activation, (xix) reducing inhibition of CTL activation, (xx) reducing inhibition of NK cell activation, (xxi) reversing T cell exhaustion, (xxii) increasing T cell response, (xxiii) increasing activity of cytotoxic cells, (xxiv) stimulating antigen-specific memory responses, (xxv) eliciting apoptosis or lysis of cancer cells, (xxvi) stimulating cytotoxic or cytostatic effect on cancer cells, (xxvii) inducing direct killing of cancer cells, and/or (xxviii) inducing complement dependent cytotoxicity and/or (xxix) inducing antibody dependent cell-mediated cytotoxicity.

In some embodiments of the invention, the immunomodulator may be administered as an adjuvant therapy during and/or following tumour resection surgery or checkpoint inhibitor therapy. This postoperative or adjuvant therapy may be used over an appropriate period of time for the patient. In some embodiments, the immunomodulator may be further administered following tumour resection surgery or checkpoint inhibitor therapy over a period of up to 2 years or more. In a preferred embodiment, the immunomodulator may be further administered over a period of 12 months post-surgery or checkpoint therapy.

In some further embodiments, following tumour resection surgery or checkpoint inhibitor therapy, the patient may be further administered one or more additional anticancer treatments or agents as described previously, with or without the immunomodulator. In some embodiments, the one or more additional anticancer treatments or agents may be administered following tumour resection surgery or checkpoint inhibitor therapy over a period of up to 2 years or more. In a preferred embodiment, the one or more additional agents or treatments may be administered over a period of 12 months post-surgery or checkpoint therapy. In some embodiments, the one or more additional anticancer treatments or agents may be administered in combination with the immunomodulator post-surgery or checkpoint therapy.

Dosages and protocols of adjuvant therapies as described herein may differ depending on what is appropriate for the particular patient and/or treatments, as would be appreciated by the skilled person. By way of example, the adjuvant administration of the immunomodulator and/or additional anticancer treatments or agents of the present invention may be administered every day, every week, every two weeks, every three weeks, every four weeks, every month, or every two months post-surgery or therapy. In some preferred embodiments, the immunomodulator and/or additional anticancer agents may be administered every four weeks post-surgery or therapy. In a most preferred embodiment, the immunomodulator and/or additional anticancer agents may be administered every four weeks for a total of 12 months post-surgery or therapy.

In other embodiments, the immunomodulator and/or additional anticancer treatments or agents may be administered in a peri-adjuvant protocol, that is, both prior to and post-surgery and/or therapy. Such peri-adjuvant protocols may include combinations of any of the neoadjuvant and/or adjuvant therapies as disclosed herein.

The peri-adjuvant administration of the immunomodulator and/or additional anticancer treatments or agents may be carried out for the length of time the cancer or tumour(s) is present in a patient or until such time the cancer has regressed or stabilized, e.g. SD or PR under RECIST 1.1 or similar, or furthermore may also be continued to be administered to the patients once the cancer or tumour has regressed or stabilised.

In further embodiments, methods of the invention include, one or more of the following: 1) reducing or inhibiting growth, proliferation, mobility or invasiveness of tumour or cancer cells that potentially or do develop metastases, 2) reducing or inhibiting formation or establishment of metastases arising from a primary tumour or cancer to one or more other sites, locations or regions distinct from the primary tumour or cancer; 3) reducing or inhibiting growth or proliferation of a metastasis at one or more other sites, locations or regions distinct from the primary tumour or cancer after a metastasis has formed or has been established, 4) reducing or inhibiting formation or establishment of additional metastasis after the metastasis has been formed or established, 5) prolonged overall survival, 6) prolonged progression free survival, 7) disease stabilisation, 8) increased quality of life.

In further embodiments, methods of the invention result in enhanced therapeutic efficacy as measured by a decrease or stabilisation of tumour size of one or more said tumours, e.g. subtotal regression as demonstrated by less than 10% vital tumour cells present in resected tumours, optionally as defined by RECIST 1.1, including stable diseases (SD), a complete response (CR) or partial response (PR) of the target tumour; and/or stable disease (SD) or partial response (PR) or complete response (CR) of one or more non-target tumours.

In a further embodiment, the invention provides a method of treating or controlling cancer comprising a primary tumour in a patient that has undergone, or is intended to undergo, tumour resection surgery, wherein said method comprises simultaneously, separately or sequentially administering to the subject, (i) a non-pathogenic non-viable Mycobacterium, (ii) tumour resection surgery, and (iii) one or more additional anticancer treatments or agents, wherein said method results in enhanced therapeutic efficacy relative to administration of non-pathogenic non-viable Mycobacterium, administration of one or more additional anticancer treatments or agents, or tumour resection surgery alone.

In a further embodiment, the invention provides a method which results in: subtotal regression as demonstrated by less than 10% vital tumour cells present in resected tumours, stable disease (SD), a complete response (CR) or partial response (PR) of the primary tumour; and/or stable disease (SD) or complete response (CR) of one or more non-target tumours, as assessed by Immune Related Response Criteria (irRC), iRECIST, RECIST 1.1, or irRECIST, preferably as assessed at 12 months post-surgery or end of therapy.

Major pathological response in more than 10%, or 20% or preferably more than 30% of tumours, can be considered clinically significant. Major pathological response is defined as complete regression or subtotal regression, preferably as demonstrated by <10% vital tumor cells present in tumour biopsy or resected tumour.

In a further embodiment, the invention provides a method which results in: (1), reducing or inhibiting formation or establishment of metastases arising from a primary tumour or cancer to one or more other sites, locations or regions distinct from the primary tumour or cancer; (2) reducing or inhibiting growth or proliferation of a metastasis at one or more other sites, locations or regions distinct from the primary tumour or cancer after a metastasis has formed or has been established, (3) reducing or inhibiting formation or establishment of additional metastasis after the metastasis has been formed or established, (4) prolonged overall survival, (5) prolonged progression free survival, (6) disease stabilisation, (7) increased quality of life, and combinations thereof.

In an embodiment of the invention, the combinations and methods disclosed herein provide a detectable or measurable improvement or overall response according to the irRC (as derived from time-point response assessments and based on tumour burden), including one of more of the following: (i) irCR—complete disappearance of all lesions, whether measurable or not, and no new lesions (confirmation by a repeat, consecutive assessment no less than 4 weeks from the date first documented), (ii) irPR—decrease in tumour burden ≥50% relative to baseline (confirmed by a consecutive assessment at least 4 weeks after first documentation). An invention method may not take effect immediately. For example, treatment may be followed by an increase in the neoplasia, tumour or cancer cell numbers or mass, but over time eventual stabilization or reduction in tumour cell mass, size or numbers of cells in a given subject may subsequently occur In an embodiment of the invention, the combinations and methods disclosed herein result in a clinically relevant improvement in one or more markers of disease status and progression selected from one or more of the following: (i): overall survival, (ii): progression-free survival, (iii): overall response rate, (iv): reduction in metastatic disease, (v): circulating levels of tumour antigens such as carbohydrate antigen 19.9 (CA19.9), carcinoembryonic antigen (CEA), prostate-specific antigen (PSA) or others depending on tumour, (vii) nutritional status (weight, appetite, serum albumin), (viii): systemic immune-inflammation index (SII) or systemic inflammation score (SIS), (ix): pain control or analgesic use, or (x): CRP/albumin ratio or prognostic nutritional index (PNI), or (xi) improved Quality of Life, or (xii), a reduction or elimination in ctDNA, preferably as assessed at 12 months post-surgery or end of therapy.

In some embodiments, the one or more markers of disease status and progression selected from the above list may be measured for monitoring of the treatment, reduction, inhibition or control protocols of the present invention. In some preferred embodiments, the one or more biomarkers may include any one or more of: prostate-specific antigen (PSA); carcinoembryonic antigen (CEA); prognostic nutritional index (PNI); systemic immune-inflammation index (SII); and systemic inflammation score (SIS).

The immunomodulator and/or one or more additional anti-cancer agents may be provided as separate medicaments for administration at the same time or at different times.

Preferably, a non-viable Mycobacterium and/or one or more additional anti-cancer agents are provided as separate medicaments for administration at different times. When administered separately and at different times, either the non-viable Mycobacterium and/or one or more additional anticancer agents may be administered first, however, it is suitable to administer a checkpoint inhibitor and/or co-stimulatory checkpoint binding agent followed by the non-viable Mycobacterium. In addition, both can be administered on the same day or at different days, and they can be administered using the same schedule or at different schedules during the treatment cycle.

In an embodiment of the invention, a treatment cycle consists of the neoadjuvant, adjuvant or peri-adjuvant administration of a non-viable Mycobacterium daily, weekly fortnightly or monthly, optionally simultaneously with an anticancer agent or checkpoint inhibitor and/or co-stimulatory checkpoint binding agent weekly, or every two weeks or every three weeks or every four weeks or more. Alternatively, the non-viable Mycobacterium is administered before and/or after the administration of the additional anticancer agent prior to, and optionally further post-surgery or therapy.

In another embodiment of the invention, the non-viable Mycobacterium is administered to the patient before and after administration of an additional anticancer agent. That is, in one embodiment, the non-pathogenic non-viable Mycobacterium is administered to the patient before and after said additional anticancer agent.

Alternatively, the administration of one or more additional anticancer agents may be performed simultaneously with the administration of the effective amounts of non-viable Mycobacterium.

The subject whom is to undergo checkpoint inhibition therapy or surgery according to the present invention may do so simultaneously, separately or sequentially with administration of the non-viable Mycobacterium.

In an aspect of the invention, the effective amount of the non-viable non-pathogenic Mycobacterium may be administered as a single dose. Alternatively, the effective amount of the non-viable Mycobacterium may be administered in multiple (repeat) doses, for example two or more, three or more, four or more, five or more, ten or more, or twenty or more repeat doses. Wherein multiple doses of Mycobacterium are administered there may be a time period of 1 week, 2 weeks, 3 weeks, 4 weeks or a combination of the aforementioned between doses.

The non-viable non-pathogenic Mycobacterium may be administered between about 8 weeks, 6 weeks or 4 weeks and/or about 1 day prior to checkpoint inhibition therapy or surgery, such as between about 4 weeks and 1 week, or about between 3 weeks and 1 week, or about between 3 weeks and 2 weeks. Administration may be presented in single or more preferably, in multiple doses. In a preferred embodiment, the immunomodulator may be administered in at least 3 doses prior to therapy or surgery.

In one embodiment of the present invention, the non-viable Mycobacterium may be in the form of a medicament administered to the patient in a dosage form.

A container according to the invention in certain instances, may be a vial, an ampoule, a syringe, capsule, tablet or a tube. In some cases, the mycobacteria may be lyophilized and formulated for resuspension prior to administration. However, in other cases, the mycobacteria are suspended in a volume of a pharmaceutically acceptable liquid. In some of the most preferred embodiments there is provided a container comprising a single unit dose of mycobacteria suspended in pharmaceutically acceptable carrier wherein the unit dose comprises about 1×10³ to about 1×10¹² organisms, or about 1×10⁶ to about 1×10⁹ organisms. In some very specific embodiments the liquid comprising suspended mycobacteria is provided in a volume of between about 0.01 ml and 10 ml, or between about 0.03 ml and 2 ml or between about 0.1 ml and 1 ml. The foregoing compositions provide ideal units for immunotherapeutic applications described herein.

Embodiments discussed in the context of a methods and/or composition of the invention may be employed with respect to any other method or composition described herein. Thus, an embodiment pertaining to one method or composition may be applied to other methods and compositions of the invention as well.

Mycobacterial compositions according to the invention will comprise an effective amount of mycobacteria typically dispersed in a pharmaceutically acceptable carrier. The phrases “pharmaceutically or pharmacologically acceptable” refers to molecular entities and compositions that do not produce an adverse, allergic or other untoward reaction when administered to an animal, such as, for example, a human, as appropriate.

In a preferred embodiment, the non-viable non-pathogenic Mycobacterium is administered via a parenteral route selected from subcutaneous, intradermal, subdermal, intraperitoneal, intravenous and intravesicular injection, or intratumoral, peritumoral, perilesional or intralesional administration. Intradermal injection enables delivery of an entire proportion of the mycobacterial composition to a layer of the dermis that is accessible to immune surveillance and thus capable of electing an anti-cancer immune response and promoting immune cell proliferation at local lymph nodes.

In another embodiment, the immunomodulator is to be administered into the skin of the patient via a microneedle device comprising a plurality of microneedles, as disclosed in WO2021/136933, incorporated herein by reference.

Other preferred microneedle devices for use according to the invention include: North Carolina State University (as described in WO2017/151727), Debioject microneedle (Debiotech, Switzerland), Micronject600 (NaoPass, Israel, as described in WO2008/047359), Nanopatch (Vaxxas, USA), SOFUSA (Kimberly-Clark, USA, as described in WO2017/189259 and WO2017/189258), Micron Biomedical's dissolving microarray, and the MIMIX dissolving, controlled release microarray (Vaxess, USA).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and immunology or related fields are intended to be within the scope of the following claims.

The invention is further described with reference to the following non-limiting Examples.

### Example 1

A study has been developed to investigate the adjuvant effects of atezolizumab in combination with a preparation of heat-killed whole cell M. obuense (IMM-101) in patients with MSI high stage III colorectal cancer ineligible for oxaliplatin-based chemotherapy, in combination with a study of the safety and efficacy of such a treatment.

Patients to be treated exhibit pathological stage III histologically-confirmed adenocarcinoma of the colon or rectum who are ineligible for, or in refusal of, oxaliplatin based adjuvant chemotherapy.

This study seeks to investigate whether the adjuvant combination of IMM-101 with atezolizumab is well-tolerated and to investigate efficacy signals of the combination, as well as measure the cell-free DNA (cfDNA) free survival rates at 12 months.

A total of 100 patients are enrolled, wherein 50 are in cohortA, and 50 are in cohort B. A sample size of N=50 is considered to provide a reasonably reliable estimate of the 12 months cfDNA free rates for the experimental combination treatments, allowing gauging of the actually observed rate against a prospectively enrolled cohort of patients with dMMR in the COLOPREDICT registry.

Cohort A receive atezolizumab at a dose of 1200 mg, administered intravenously, every 3 weeks for 12 months. Cohort B receive atezolizumab at a dose of 1200 mg, administered intravenously, every 3 weeks for 12 months, in combination with IMM-101 every 2 weeks for 3 doses, and subsequently every 4 weeks for a total of 12 months.

IMM-101 is administered as a single 0.1 mL intradermal injection of IMM-101 (10 mg/mL, a sterile suspension of heat-killed M. obuense NCTC 13365 in borate buffered saline) into the skin overlying the deltoid muscle, with the arm being alternated between each dose. The Investigator will have been appropriately trained a priori in the technique of intradermal injection.

Previous clinical experience with IMM-101 has suggested that this dose is safe and well tolerated. The skin reaction that develops at the site of injection is characterised by erythema, local swelling and occasionally mild ulceration. All symptoms are to be expected given the known pharmacology of the product and previous clinical experience. Furthermore, data from safety and tolerability studies with IMM-101 have revealed that skin reactions resolve satisfactorily over time and do not impair daily activity.

The first dose of IMM-101 administered to each patient in the study is followed by vital signs monitoring for at least 2 hours under medical supervision with resuscitation facilities available as a precautionary measure.

The treatment regimen will be 1 dose of IMM-101 given every 2 weeks for the first 3 doses followed by a rest period of 4 weeks, then one dose every 2 weeks for the next 3 doses. This will be followed by a dose every 4 weeks thereafter with a window of +/−2 days allowed. The initial dose of IMM-101 prior to surgery is intended to be 1.0 mg followed by 0.5 mg, and/or the initial dose of IMM-101 following surgery is intended to be 1.0 mg followed by 0.5 mg.

Atezolizumab is administered at 1200 mg intravenously every 3 weeks for 12 months.

Treatment for patients in both cohorts is continued for 12 months, or until disease progression (as assessed by Response Evaluation Criteria in Solid Tumours [RECIST] 1.1) subject to the following qualifications: unacceptable side-effects, the investigator's decision to discontinue treatment, withdrawal of patient consent, or 18 months of IMM-101 treatment, whichever is the sooner. Patients with a complete response maintained over 2 scans should continue treatment unless the investigator considered this not in the patient's best interest. Patients in cohorts A and B who have documented disease progression may continue treatment on study if they have a clinical benefit and no decline in performance status, no clinically relevant adverse effects with the study treatment as determined by the investigator, or are not deemed to require alternative treatment.

### Example 2

An explorative sub-study to Example 1 has been developed to assess the efficacy of neoadjuvant atezolizumab with or without IMM-101 in patients with MSI high clinical stage III colorectal cancer based on preoperative CT scans in terms of pathological complete (pCR) or subtotal (<10% vital tumour cells) regression after 6 weeks treatment, as well as assessing the safety and tolerance of such a treatment protocol.

20 of the 100 patients of Example 1 with clinical stage III disease based on a preoperative CT scan are randomised 1:1 to receive neoadjuvant atezolizumab and/or IMM-101 for 6 weeks. After resection, these patients will receive the adjuvant therapy of Example 1 for an additional 12 months.

Cohort A (10 patients) are administered atezolizumab at 1200 mg, intravenously, every 3 weeks for 6 weeks preoperatively, and 12 months after resection. Cohort B (10 patients) are administered IMM-101 at 0.1 ml of 10 mg/ml solution, intradermally, every 2 weeks for three doses preoperatively, and subsequently every 4 weeks, for a total of 12 months in the adjuvant setting of Example 1.

### Example 3

A study has been developed to investigate the adjuvant effects of atezolizumab in combination with a preparation of heat-killed whole cell M. obuense (IMM-101) in patients with MSI-H/dMMR stage III colorectal cancer for whom oxaliplatin regiments are not a viable treatment option.

Patients to be treated have an ECOG status of 0-2, have undergone R0 tumor resection, and exhibit MSI-high and dMMR, pathological stage III, histologically-confirmed adenocarcinoma of the colon or rectum who are ineligible for, or in refusal of, oxaliplatin based adjuvant chemotherapy.

This study seeks to investigate whether the adjuvant combination of IMM-101 with atezolizumab is well-tolerated and to investigate efficacy signals of the combination, as well as determine whether the adjuvant combination of IMM-101 with atezolizumab can significantly improve disease-free survival rate at 3 years, as well as estimating the ctDNA-free rate defined as the proportion of patients without detectable ctDNA after 12 months of adjuvant treatment.

A total of 100 patients are enrolled, wherein 50 are in cohortA, and 50 are in cohort B. A sample size of N=50 is considered to provide a reasonably reliable estimate of the 3-year disease-free survival rate for the experimental combination treatments, allowing gauging of the actually observed rate against a prospectively enrolled cohort of patients with stage III dMMR tumors in the COLOPREDICT registry.

Cohort A receive atezolizumab, in an adjuvant setting, at a dose of 840 mg, administered intravenously, every 2 weeks for 12 months.

Cohort B receive, in an adjuvant setting, one initial dose of 1.0 mg IMM-101 administered intradermally at day 14±2 days before the start of atezolizumab treatment. Then, patients are administered atezolizumab at a dose of 840 mg, administered intravenously every 2 weeks for 12 months, in combination with IMM-101 at a dose of 0.5 mg intradermally, every 2 weeks for one month, and subsequently at a dose of 0.5 mg intradermally every 4 weeks for a total of 12 months.

The dose volume of IMM-101 is initially 0.1 mL (1.0 mg), followed by 0.05 mL (0.5 mg), administered intradermally into the skin overlying the deltoid muscle, with the arm being alternated between each dose. The Investigator will have been appropriately trained a priori in the technique of intradermal injection.

Previous clinical experience with IMM-101 has suggested that this dose is safe and well tolerated. The skin reaction that develops at the site of injection is characterised by erythema, local swelling and occasionally mild ulceration. All symptoms are to be expected given the known pharmacology of the product and previous clinical experience. Furthermore, data from safety and tolerability studies with IMM-101 have revealed that skin reactions resolve satisfactorily over time and do not impair daily activity,

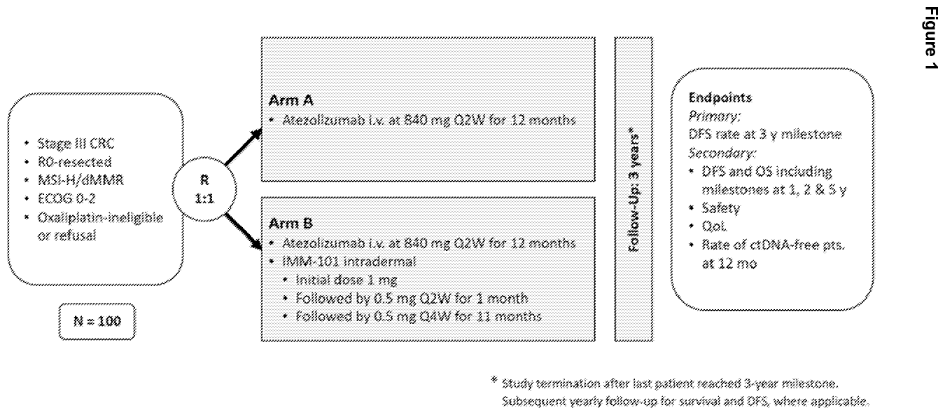

The first dose of IMM-101 administered to each patient in the study is followed by vital signs monitoring for at least 2 hours under medical supervision with resuscitation facilities available as a precautionary measure. Patients are followed up for three years following the last dose of therapy where the primary endpoint is an assessment of the DFS rate at the 3-year milestone, with secondary endpoints to include DFS/OS at 1, 2 and 5 years, safety, QoL and rate of ctDNA free patients at 12 months following the end of therapy—see FIG. 1.

Patient inclusion criteria include: hemoglobin of at least 9 g/dL; AST (SGOT)/ALT (SGPT) value of ≤2.5; with endpoints to further include: death from any cause 3 years after randomization.

### Example 4

An explorative sub-study to Example 3 has been developed to assess the efficacy of perioperative/peri-adjuvant atezolizumab with IMM-101 in patients with MSI-high/dMMR clinical stage Ill colorectal cancer for whom oxaliplatin regimens are not a viable treatment option (or refused), in terms of pathological complete (pCR) or subtotal (<10% vital tumour cells) regression after 5 weeks treatment, and in terms of disease-free survival and overall survival.

A total of 20 patients are enrolled, wherein patients to be treated exhibit MSI-high/dMMR clinical stage III histologically-confirmed adenocarcinoma of the colon or rectum who are ineligible for, or in refusal of, oxaliplatin based adjuvant chemotherapy, have an ECOG status of 0-2 and present with a resectable primary tumor. After resection, these patients will receive the adjuvant therapy of Example 3, Cohort B, for an additional 12 months.

Specifically, enrolled patients receive atezolizumab at a dose of 1200 mg, administered intravenously, 28 days and 7 days prior to tumor resection surgery, in combination with IMM-101 at a dose of 1.0 mg, administered intradermally, 35 days prior to tumor resection surgery, followed by IMM-101 administered at a dose of 0.5 mg 21 days and 5 days prior to resection surgery.

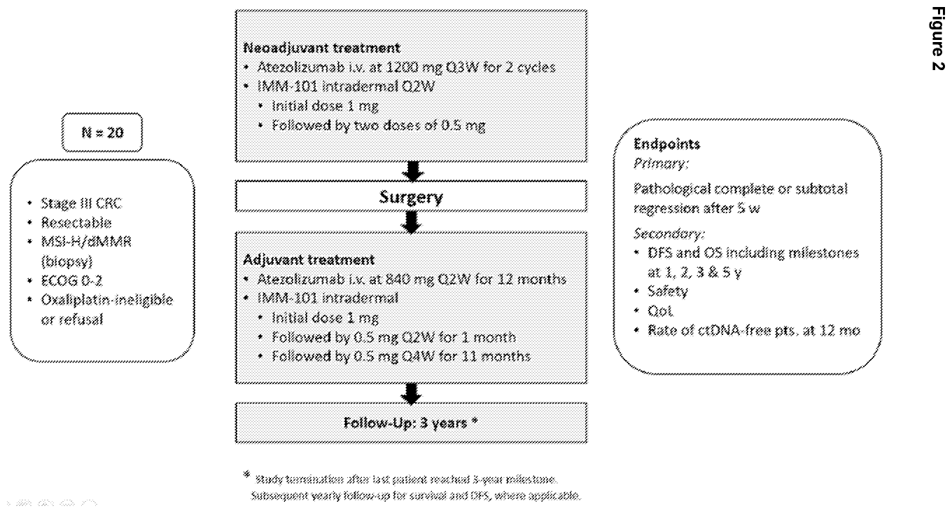

Within 70 days after resection surgery, patients will receive one initial dose of 1.0 mg of IMM-101 administered intradermally at day 14±2 days before the start of atezolizumab treatment. This is followed by administration of atezolizumab at a dose of 840 mg, administered intravenously, every 2 weeks for 12 months, in combination with IMM-101 at a dose of 0.5 mg, every 2 weeks for one month, and subsequently every 4 weeks for a total of 12 months, as per the adjuvant setting of Example 3, Cohort B. Patients are followed up for three years following the last dose of therapy where the primary endpoint is an assessment of the DFS rate at the 3-year milestone, with secondary endpoints to include DFS/OS at 1, 2 and 5 years, safety, QoL and rate of ctDNA free patients at 12 months following the end of therapy—see FIG. 2.

1-37. (canceled)

38. A method of treating or controlling cancer comprising a primary tumour in a patient that has undergone, or is intended to undergo, tumour resection surgery, wherein said method comprises simultaneously, separately or sequentially administering to the subject, (i) a non-pathogenic non-viable Mycobacterium, (ii) tumour resection surgery, and (iii) one or more additional anticancer treatments or agents, wherein said method results in enhanced therapeutic efficacy relative to administration of non-pathogenic non-viable Mycobacterium, administration of one or more additional anticancer treatments or agents, or tumour resection surgery alone.

39. The method according to claim 38, wherein said method results results in: subtotal regression as demonstrated by less than 10% vital tumour cells present in tumour biopsy or resected tumours, stable disease (SD), a complete response (CR) or partial response (PR) of the primary tumour; and/or stable disease (SD) or complete response (CR) of one or more non-target tumours, as assessed by Immune Related Response Criteria (irRC), iRECIST, RECIST 1.1, or irRECIST.

40. The method according to claim 39, wherein said method results in (1), reducing or inhibiting formation or establishment of metastases arising from a primary tumour or cancer to one or more other sites, locations or regions distinct from the primary tumour or cancer; (2) reducing or inhibiting growth or proliferation of a metastasis at one or more other sites, locations or regions distinct from the primary tumour or cancer after a metastasis has formed or has been established, (3) reducing or inhibiting formation or establishment of additional metastasis after the metastasis has been formed or established, (4) prolonged overall survival, (5) prolonged progression free survival, (6) disease stabilisation, (7) increased quality of life, and combinations thereof.

41. The method according to claim 38, wherein said one or more additional anticancer treatments or agents is selected from adoptive cell therapy, surgical therapy, chemotherapy, radiation therapy, hormonal therapy, checkpoint inhibitor therapy, small molecule therapy, receptor kinase inhibitor therapy, hyperthermia treatment, phototherapy, radiofrequency ablation therapy (RFA), anti-angiogenic therapy, cytokine therapy, cryotherapy, biological therapy, HDAC inhibitor, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonists, STING agonists, mifamurtide, cancer vaccines, and combinations thereof.

42. The method according to claim 38, wherein the cancer is selected from the group consisting of bladder cancer, prostate cancer, liver cancer, renal cancer, lung cancer, breast cancer, colorectal cancer, colon cancer, rectal cancer, pancreatic cancer, brain cancer, hepatocellular cancer, lymphoma, leukaemia, gastric cancer, cervical cancer, ovarian cancer, thyroid cancer, melanoma, head and neck cancer, skin cancer and soft tissue sarcoma and/or osteosarcoma, pancreatic ductal adenocarcinoma (PDAC) and pancreatic neuroendocrine tumours (PNET).

43. The method according to claim 42, wherein said cancer is metastatic.

44. The method according to claim 38, wherein said cancer is clinically defined by a TNM staging criteria, in which the patient has a primary tumour (T) of T1 to T4, and/or a Node designation of N0, N1 or N2, or wherein said cancer is clinically defined as being Stage I, Stage II or Stage III.

45. The method according to claim 44, wherein said patient has no evidence of metastasis (M0), as defined by a TNM staging criteria.

46. The method according to claim 38, wherein non-pathogenic non-viable Mycobacterium is selected from M. vaccae, the strain deposited under accession number NCTC 11659 and associated designations SRL172, SRP299, IM-201, DAR-901, and the strain as deposited under ATCC 95051; M. obuense, M. paragordonae (strain 49061), M. parafortuitum, M. paratuberculosis, M brumae, M aurum, M. indicus pranii, M. w, M. manresensis, M. kyogaense (as deposited under DSM 107316/CECT 9546), M. phlei, M. smegmatis, M. tuberculosis Aoyama B or H37Rv, RUTI, BCG, VPM1002BC, SMP-105, Z-100, the strain of Mycobacterium obuense deposited under the Budapest Treaty under accession number NCTC 13365 and combinations thereof.

47. The method according to claim 46, wherein the non-pathogenic non-viable Mycobacterium is M. obuense NCTC 13365 or a fraction, fragment, sub-cellular component, lysate, homogenate, sonicate, or substantially in whole cell form.

48. The method according to claim 38, wherein the immunomodulator is administered in a dose comprising 10³-10⁹ cells, or 0.0001 to 1.0 mg.

49. The method according to claim 38, wherein the immunomodulator is administered prior to tumour resection surgery and/or administration of said one or more additional anticancer treatments or agents for at least 1 to 3 doses or for 3 doses.

50. The method according to claim 38, wherein the immunomodulator is administered following tumour resection surgery or administration of said one or more additional anticancer treatments or agents optionally wherein said administration is over a period of 12 months or more.

51. The method according to claim 38, wherein said one or more agents are selected from: bevacizumab, cyclophosphamide, methotrexate, 5-fluorouracil, doxorubicin, mustine, vincristine, procarbazine, prednisolone, bleomycin, vinblastine, dacarbazine, etoposide, cisplatin, epirubicin, capecitabine, leucovorin, folinic acid, carboplatin, oxaliplatin, gemcitabine, FOLFIRINOX, FOLFOX, mFOLFIRINOX, NALIRIFOX, paclitaxel, nab-paclitaxel, pemetrexed, irinotecan, temozolomide and combinations thereof, wherein said one or more additional anticancer treatments or agents is administered intratumorally, intraarterially, intravenously, intravascularly, intrapleuraly, intraperitoneally, intratracheally, intranasally, pulmonarily, intrathecally, intramuscularly, endoscopically, intralesionally, percutaneously, subcutaneously, regionally, stereotactically, orally or by direct injection or perfusion.

52. The method according to claim 38, wherein said administration of one or more additional anticancer treatments or agents is a checkpoint inhibitor therapy selected from ipilimumab, nivolumab, pembrolizumab, azetolizumab, BI 754091 (anti-PD-1), bavituximab, bintrafusp alfa, durvalumab, dostarlimab, tremelimumab, spartalizumab, avelumab, sintilimab, toripalimab, prolgolimab, tislelizumab, camrelizumab, MGA012, MGD013(tebotelimab), MGD019, enoblituzumab, MGD009, MGC018, MEDIO680, miptenalimab, nimotuzumab, PDR001, FAZ053, TSR022, MBG453, relatlimab (BMS986016), LAG525 (IMP701), IMP321 (Eftilagimod alpha), REGN2810 (cemiplimab), REGN3767, pexidartinib, LY3022855, FPA008, BLZ945, GDC0919, epacadostat, emactuzumab, FPA150, indoximid, BMS986205, CPI-444, MEDI9447, PBF509, FS118, lirilumab, Sym023, TSR-022, A2Ar inhibitors, NKG2A, and combinations thereof.

53. The method according to claim 52, wherein one or more checkpoint inhibitors are each administered in a sub-therapeutic amount and/or duration.
